(19) United States
(12) Patent Application Publication
(10) Pub. No.: US 2018/0161022 A1
(43) Pub. Date: Jun. 14, 2018

(54) ORTHOPAEDIC MEDICAL DEVICE

(71) Applicant: University of Leeds, Leeds, Yorkshire (GB)

(72) Inventors: Thomas BABOOLAL, Leeds (GB); Elena A. JONES, Leeds (GB); Denis McGONAGLE, Leeds (GB)

(21) Appl. No.: 15/508,878

(22) PCT Filed: Sep. 4, 2015
(86) PCT No.: PCT/GB2015/052554

§ 371 Date: Mar. 3, 2017

(30) Foreign Application Priority Data
Sep. 4, 2014 (GB) 1415653.3

(51) Int. Cl. A61B 10/02

(52) U.S. Cl. CPC A61B 10/0233; A61B 2010/0216

(57) ABSTRACT

This present invention provides an orthopaedic medical device, in particular an arthroscopic aid, for stimulating the release of cells and/or encouraging migration of cells to a surgical site or an injury site. The invention includes methods of increasing a cell population at a surgical site or injury, a method of improving surgical outcome of synovial joint procedures and methods of delivering intra-operative minimally manipulated cells to a synovial joint.

This present invention relates to an orthopaedic medical device, in particular an arthroscopic aid, for stimulating the release of cells and/or encouraging migration of cells to a surgical site and/or a site of injury. The invention includes, inter alia, methods of increasing a cell population at a surgical site and/or a site of injury, a method of improving surgical outcome of synovial joint procedures and methods of delivering intra-operative minimally manipulated autologous or allogeneic cells to a synovial joint.

### BACKGROUND

Synovial joints, or diarthrosis, are the most abundant types of joints in mammals and they include gliding joints, hinge joints, pivot joints, condyloid joints, ball and socket joints and compound joints such as the knee. Synovial joints are characterised by presence of an inner synovial membrane lining the joint cavity and adjacent surrounding capsules. The synovial membrane produces a lubricating synovial fluid that bathes the synovial cavities and nourishes the cartilage that lines the ends of the bones.

Attrition to the cartilage lining of joints leads to “wear and tear” or osteoarthritis (OA). The non-surgical treatments for OA include physiotherapy and the non-steroidal anti-inflammatory drugs (NSAIDs) which are a type of painkiller that is usually recommended for minor to moderate cases of damage. There is a dearth of treatments available for OA and ultimately the only effective therapy may be a prosthesis, such as a knee or hip replacement device.

Unfortunately, cartilage tissue has a limited propensity for repair, due in part to the avascular nature of cartilage itself and therefore its limited nutrient supply, but also due to deficiencies in medical and surgical treatment options to elicit effective repair. To overcome these shortcomings cell-based therapies, in particular those utilizing mesenchymal stem cells (MSCs), are an attractive prospect especially in the large group of patients not responding to pain killers but with disease not severe enough to warrant total joint replacement. These adult stem cells are relatively easy to isolate from a variety of tissues (including bone marrow, bone, periosteum, adipose tissue, synovium and synovial fluid) and have the ability to differentiate into, for example, cartaliginous and connective tissue. MSCs are thus ideal candidates for therapeutic use in many musculoskeletal settings and offer novel regenerative medicine approaches for joint repair.

It is known from the prior art that bone marrow and synovial fluid provide a good source of MSCs (Jones et al, Arthritis and Rheumatism, 2002; 46(12):3349-60 and Jones et al, Arthritis and Rheumatism, 2004; 50(3):817-27). The discovery of a resident population of MSCs within synovial fluid was the first description of a potentially reparative population having direct access to superficial cartilage and joint structures. Injection of expanded MSCs into synovial fluid led to the integration of these MSCs into damaged menisci and ligament in small animal models (Murphy et al Arthritis and Rheumatism, 2003; 48(12):3464-74 and Mizuno et al J. Med. Sci Dental Sci 2008; 55(1):101-11), indicating that injection of expanded MSCs in the synovial fluid is an effective form of administration. More recently it has been shown that expanded MSCs incubated for as little as 10 minutes in full-thickness cartilage defects is enough to elicit cartilage repair in pigs (Nakamura et al Cytotherapy 2012: 14(3):327-38). In these and other settings, the prior art emphasis has been on the administration of extracted and culture expanded MSCs of autologous or allogeneic origin and that such cells may be capable of joint repair.

A medical device that would allow a practitioner to increase the number of mesenchymal stem cells (MSCs) in situ during an operative procedure without the need to remove tissue from the body, transport it asceptically to the laboratory, digest the tissue, culture expand MSCs for several weeks and finally transfer them back to the patient for a second operative procedure would offer immediate benefit to patients, medical practitioners and health economists alike. Therefore a medical device that could release mesenchymal stem cells (MSCs) in situ during an operative procedure would represent a novel one stage stem cell procedure for tissue regeneration.

### BRIEF SUMMARY OF THE DISCLOSURE

According to a first aspect of the invention there is provided an orthopaedic medical device comprising a head portion, a neck portion, a stem portion and a handle portion, the head portion having a first surface that is abrasive and provided with at least one abrasive, flexible projecting element that is non-circular in cross-section and an opposing second surface, the second surface being non-abrasive or smooth.

Preferably, the abrasive surface of the head portion comprises a plurality of flexible projecting elements each element having a tip and a base. The projecting elements may be formed integrally at their bases with the abrasive surface of the head portion or moulded thereto or they may be attached thereto at their bases by any suitable means, such as a biocompatible adhesive.

Preferably, the flexible projecting elements in some embodiments are rigid about their base region and flexible at their tip region or vice versa. In some embodiments of the invention the projecting elements are moveable about their base and flexible along their length. In all embodiments the projecting elements are flexible for at least a portion if not all of their length.

Preferably, the projecting elements are in the form of a bristle, brush, fin, prong, hair, spike, quill, fiber or rod.

Preferably, the tip or distal end of the projecting elements, that is to say the end not associated with the base of the head portion, may be flat, tapered, conical or profiled.

Preferably, the projecting elements are be perpendicular with respect to the abrasive surface of the head portion, that is to say that they are at 90° with respect to the abrasive surface and effectively stand upright. However, in some embodiments they may be angled with respect to the abrasive surface at an angle of between 35° to less than 90°. In some embodiments only a proportion of the projecting elements are perpendicular whilst others are angled.

Preferably, the projecting elements are arranged in columns and rows. The columns are defined along the length of the abrasive surface whereas rows are defined across the width of the abrasive surface. Preferably there are between 2 to 20 projecting elements in any one column and between 2 to 10 projecting elements in any one row.

Preferably, the projecting elements in cross section are non-circular and are for example shaped as squares, rectangles, diamonds, circles, triangles, stellate (star-shaped), rhombic, oval, chevrons, “V” shaped, “W” shaped, contiguous wave, pentagons, hexagons or other multiple sides shapes. The pattern of the projecting arranged on the brush head may be in a regular pattern or maybe in a random pattern. A particularly preferred embodiment is an arrangement of rectangular cross sectional elements arranged so as to be at an angle of 90° with respect to a neighbouring element.

Preferably, the abrasive surface may comprise projecting elements that are all of the same cross-sectional shape or they may be of a variety of two or more mixed cross-sectional shapes.

Preferably, at least a proportion of the projecting elements are rectangular, diamond or rhombic in cross section.

Preferably, the projecting elements are of equal length however in some embodiments they may be of a greater length at either the proximal end of the abrasive surface (end closest to the stem portion) than the distal end (end of the abrasive surface furthest away from the stem portion). Alternatively they may taper in highest length to lowest length or lowest length to highest length so that a peak or tough of length lies somewhere between the distal and proximal ends of the abrasive surface.

Preferably, the density of the projecting elements is in the range from about 1 to 1000 projecting elements/cm². Optionally, the range may be from about 10, 20, 50, 100, 200, 300, 400, 500, 700, or 800 projecting elements/cm² to about 20, 50, 100, 200, 300, 400, 500, 700, 800 or 1000 projecting elements/cm² (or any range and integer therein between).

Preferably, the total distance of the underside or base of the non-abrasive side of the head portion to the tip or apex of the projecting element is between 3.00 to 20.00 mm, and more preferably is between 6.00 to 12.00 mm and more preferably still is between 7.00 to 10.00 mm or any tenth or one hundredth integer of 0.00 to 0.99 and therebetween.

In the instance of the medical device of the present invention being used in an arthroscopic procedure on, for example, a knee of a human subject, it is desirable that the height of the base of the of the non-abrasive side of the head portion to the tip of the projecting elements is around 7.00 mm since the portal of an arthroscope is typically around 7.00 mm. Conversely, in the instance of the medical device of the present invention being used in an arthroscopic procedure on, for example, a shoulder of a human subject, it is desirable that the height of the base of the of the non-abrasive side of the head portion to the tip of the projecting elements is around 10.00 mm since the portal of an arthroscope is typically around 10.00 mm. The distance between the base of the non-abrasive side of the head portion to the tip of the projecting elements may be longer for hip procedures.

Preferably, the length of the projecting element from its base i.e. where it is attached to the upper side of the non-abrasive side of the head portion and begins to for the abrasive side of the head portion, to tip is in the range from about 1.00 mm to about 5.00 mm or any tenth or one hundredth integer of 0.00 to 0.99 and therebetween.

Preferably the projecting elements comprises a contiguous element comprising a proximal portion, a middle portion and a distal portion. Preferably, the length of the proximal portion i.e. the portion attached to or associated with the upper surface of the non-abrasive surface of the head portion is between 0.10 to 3.00 mm or any integer therebetween.

Preferably the length of the middle portion i.e. the portion between the proximal and distal end of the projecting element is between 0.10 to 3.00 mm or any integer therebetween.

Preferably the length of the distal portion i.e. the tip portion and the portion furthest away from the base of the guide portion and the portion which in use, is in contact with the synovial membrane, is between 0.10 to 3.00 mm or any integer therebetween.

In some embodiments of the invention the proximal portion, middle portion and distal portion the projecting element are constructed of the same material or are constructed of different materials so that the flexibility of the projecting element may be different along its length.

Preferably the distal portion of the projecting element may be profiled so that it provides a point or apex.

Preferably the abrasive surface of the head portion comprises a plurality of projecting elements of differing length, preferably the projecting elements of a shorter length are positioned around the periphery of the abrasive surface so that the projecting elements of a longer length are concentrated in the centre of the abrasive surface. Preferably, the projecting elements at the periphery of the abrasive surface increase incrementally in length to the tallest projecting elements at the centre of the abrasive surface. In one particular embodiment of the invention the length of the projecting elements at the periphery are between 1.00 to 2.00 mm and the projecting elements of the central region of the abrasive surface are between 3.00 to 4.00 mm. It will be appreciated that the length of projecting elements and depth of the non-abrasive surface may be of any dimension providing that the maximal distance from the underside or base of the non-abrasive side of the head portion to the tip or apex of the projecting element is commensurate with the portal of the scoping device.

Preferably, the projecting elements are constructed of any one or more of the following materials: acetal, Teflon (polytetrafluoroethylene, polyester, nylon, polyethylene, polyurethane, polypropylene, polycarbonate, silicone rubber, polyether ether ketone (PEEK) or any combination thereof.

Preferably, the second surface opposing is non abrasive and devoid of projecting elements so that, in use and in the instance of it being used in an arthroscopic procedure, the second surface is able to slide or glide over the cartilage with reduced friction. In this way, advantageously, the cartilage is not damaged by the medical device.

Preferably, the length of the head portion is appropriate for the orthopaedic operation being performed. By way of example, but not for the purposes of limitation, the head portion may be between 5 to 25 mm in length, and more preferably is between 10 and 20 mm in length and more preferably still is about 15 mm in length. These recited lengths may be considered appropriate for example for an orthopaedic operation that is carried out on the knee of a human subject. The skilled person would be readily identify lengths that are suitable for use in a specific orthopaedic operation.

Preferably, the head portion and the guide portion may be formed integrally or alternatively the head portion may be detached from the guide portion.

Preferably, the head, neck and stem portions are formed from the same material. Alternatively, the head portion and the stem portion are formed from different materials. Optionally, each portion (e.g. head portion or neck portion or stem portion) may be formed from one or more materials. Constructing the portions of the device in different materials allows for varying degrees of rigidity along the length of the device and can be selected according to the surgical procedure in which it has intended use.

Preferably, the distal tip of the head portion is curved or profiled so as to reduce the likelihood of damage to tissue.

Preferably, the stem portion is rigid. Rigidity of the stem portion allows it to act as a lever enabling the device to reach more constricted areas within the joint and allows the surgeon more control of the device in situ.

Preferably, the stem portion is between 100 to 400 mm in length and more preferably is between 150 to 250 mm in length. It will be appreciated that the stem portion length is dependent on the surgical procedure in which it has intended use. In some embodiments of the invention the stem portion maybe telescopic so that the length may be adjustable according to a surgeon's or patients' requirements.

Preferably, the neck portion is angled. Angling at the distal end of the device is to assist easy and trauma free passage over cartilage and also to permit access to all parts of the joint cavity.

Preferably, the neck portion is angled with respect to horizontal axis of the stem portion by between 5 to 30° , and more preferably by 10 to 20 ° and more preferably still by 15 ° . In some embodiments of the invention the neck portion may be hinged with the stem portion.

Preferably, the head portion of the medical device of the present invention is provided with one or more additional channels for the delivery of a fluid to the surgical site and/or suction of fluid therefrom. Preferably, at least one of the additional channels is pressurized so as to deliver fluid for irrigation whereas at least one other channel is under vacuum for aspirating liquid and cellular debris away from the site. In some embodiment of the invention the aspirated cells can be filtered either within the medical device or remotely therefrom by, for example and without limitation, centrifugal forces or filtration and re-introduced to the site of injury or site of the operative procedure or used to seed scaffolds for subsequent implantation or research. In this embodiment of the invention the released cells and in particular MSCs are “sucked up” and collected and optionally concentrated for subsequent re-introduction into a recipient or for seeding a scaffold, for example and without limitation a cartilage, osteochondral, meniscal or ligament scaffold. In some embodiments cells are collected for allogeneic uses for example cells can be harvested from a young donor to be given to an older recipient.

Preferably the irrigation channel and/or aspiration channel is provided with a number of ports adjacent the base of the projecting elements. Alternatively the channels may have single entry and exit ports.

Preferably, the stem portion of the medical device includes a marker region to allow the surgeon to ascertain either the orientation of the medical device and/or to ascertain when the head portion is in position at the appropriate surgical site.

Preferably, the medical device of the present invention is associated with an external power source that can provide vibrational or oscillatory movement to the head region such as ultrasonication.

According to a second aspect of the invention there is provided use of the device of the first aspect of the invention in arthroscopic procedures.

According to a third aspect of the invention there is provided an arthroscope or other synovial joint scoping device and attached thereto the medical device of the first aspect of the invention.

According to a fourth aspect of the invention there is provided a method of stimulating release of mesenchymal stem cells (MSCs) cells and/or encouraging migration of mesenchymal stem cells (MSCs) and/or recruiting mesenchymal stem cells (MSCs) in situ to a surgical site or site of injury during an operative procedure on a synovial joint, the method comprising contacting synovial membrane or adjacent tissue with a device having at least one abrasive surface.

It is understood that the synovial membrane is only one or two cells in thickness in healthy individuals but may be thicker in osteoarthritic or injured individuals and is porous therefore cells may also be released from the subsynovium or from fatty or fibrofatty synovium of the fat pad closely juxtaposed to the synovium. In this respect all aforementioned tissues are considered as “adjacent tissues” to the synovial membrane.

Preferably, the device is that according to the first aspect of the invention.

Preferably the synovial joint is selected from the group comprising gliding joints, hinge joints, pivot joints, condyloid joints, ball and socket joints and compound joints.

Preferably, the synovial joint is a compound joint and more preferably is a knee joint.

Preferably, the operative procedure is selected from the group comprising arthroscopy, meniscectomy, chondroplasty, cruciate ligament repair, knee replacement, mosaicaplasty and meniscus repair.

Knee arthroscopy is commonly performed for treating meniscus injury, reconstruction of the anterior cruciate ligament and for cartilage microfracturing. Knee arthroscopy can be used in any one of the following situations, which are included within the scope of the present invention:
(i) to remove or repair torn meniscal cartilage which cushions the space between the bones in the knee;
(ii) to reconstruct a torn anterior cruciate (ACL) or posterior cruciate ligament (PCL);
(iii) to trim torn pieces of articular cartilage;
(iv) to remove loose fragments of bone or cartilage;
(v) to remove inflamed synovial tissue;
(vi) to repair misalignment of the patella;
(vii) to aid repair of osteochondral defects including osteochondritis dessicans;
(viii) to treat arthritis in younger patients;
(ix) for diagnostic or investigational purposes;
(x) mosiacaplasty grafting by transferring one or more cylindrai osteochondral autografts from a low weight-bearing area of the knee towards the defective site;
(xi) to harvest MSCs from a donor.

According to a fifth aspect of the invention there is provided a method of increasing a cell population of mesenchymal stem cells (MSCs) in situ at a surgical site or injury site during an operative procedure on a synovial joint, the method comprising contacting synovial membrane and adjacent tissue with a device having at least one abrasive surface.

Preferably, fifth aspect of the invention includes all the features of the fourth aspect of the invention.

According to a sixth aspect of the invention there is provided a method of delivering intra-operative minimally manipulated autologous mesenchymal stem cells (MSCs) to a surgical site or site of injury during an operative procedure on a synovial joint, the method comprising contacting synovial membrane and adjacent tissue with a device having at least one abrasive surface.

Preferably, sixth aspect of the invention includes all the features of the fourth aspect of the invention.

According to a seventh aspect of the invention there is provided a method of improving surgical outcome of a synovial joint surgical procedure, the method comprising contacting synovial membrane tissue with a device having at least one abrasive surface during the surgical procedure.

Preferably, seventh aspect of the invention includes all the features of the fourth aspect of the invention.

According to an eighth aspect of the invention there is provided a method of delivering intra-operative minimally manipulated allogeneic mesenchymal stem cells (MSCs), collected from a donor, to a surgical site during an operative procedure on a synovial joint, the method comprising contacting synovial membrane tissue of an allogeneic donor with a device having at least one abrasive surface and collecting said cells.

Preferably, the eighth aspect of the invention includes all the features of the fourth aspect of the invention.

According to an ninth aspect of the invention there is provided a method of delivering intra-operatively a concentrate of minimally manipulated allogeneic or autologous mesenchymal stem cells (MSCs), collected from a patient or donor, to a surgical site during an operative procedure on a synovial joint, the method comprising contacting synovial membrane tissue of with a device having at least one abrasive surface and collecting said cells, preparing a concentrate of said cells and introducing said cell concentrate to the surgical site.

Preferably, ninth aspect of the invention includes all the features of the fourth aspect of the invention.

In a yet further aspect of the invention there is provided a method of allogeneic harvesting of MSCs for use in re-cellularising a native or synthetic scaffold for subsequent implantation into a recipient.

In this embodiment of the invention cells may be harvested and optionally concentrated prior to seeding an implantable device for use inside, adjacent or external to a synovial joint or for ligament and meniscal repair.

In a yet further aspect of the invention there is provided use of the device of the present invention in conjunction with microfracture, whereby the subchondral bone is breeched in order to induce bleeding and clot formation and MSCs from bone marrow are entrapped within the clots, which subsequently forms repair tissue.

The features ascribed to one aspect of the invention apply mutatis mutandis to each and every aspect of the invention.

The device and methods of the present invention provide a means of increasing MSC number within the joint. This increase in the number of MSCs will allow an opportunity for MSCs to more effectively interact with all damaged joint structures including cartilage, meniscus, ligaments and exposed bony surfaces.

### BRIEF DESCRIPTION OF THE DRAWINGS

Embodiments of the invention are further described hereinafter with reference to the accompanying drawings, in which:

FIG. 1 shows a schematic representation of the present invention with different guide and head embodiments.

FIG. 2 shows one embodiment of the medical device of the present invention.

FIG. 3 shows one embodiment of the head design of the medical device of the present invention.

FIG. 4 shows an alternative embodiment of the head design of the medical device of the present invention.

FIG. 5 shows an alternative embodiment of the head design of the medical device of the present invention.

FIG. 6 shows an alternative embodiment of the head design of the medical device of the present invention.

FIG. 7 shows an alternative embodiment of the head design of the medical device of the present invention.

FIG. 8 shows the head design of the medical device of the present invention incorporating irrigation and aspiration channels.

FIG. 9A shows an example colony-forming unit-fibroblasts (CFU-F) assay to measure MSCs from 50 mL PBS washout of a normal cadaveric knee. FIG. 9B shows the corresponding CFU-F assay for released cells from the same cadaveric knee as in (A). FIG. 9C shows cumulative CFU-F assay data from n=10 synovium showing a median increase of 2.7-fold in the number of colonies after release of cells from superficial synovium using the device. ** indicates p=0.002.

FIG. 10A shows example data showing the number of CFU-F colonies generated after 14 days from four different embodiments (brush designs C1, C2, C3 and C4). FIG. 10B shows the comparative data between brushes C1 (circular in cross-section) and C2 (square in cross-section) and FIG. 100 shows the comparative date between brushes C3 (triangular shaped in cross-section) and C4 (diamond in cross-section). Each data point represents average colony number from duplicates CFU-F plates as in FIG. 9. Matched samples are shown in B and C by the solid line. * indicates p=0.03, n=6.

FIG. 11A shows tri-lineage differentiation (chondrogenesis, osteogenesis and adipogenesis) of MSCs released from the superficial synovium by mechanical agitation of the device of the present invention. FIG. 11B shows the total GAG pg/pellet against MSC chondrocyte culture for three donors. FIG. 110 shows the total calcium/ml against MSC osteocytes culture for three donors. FIG. 11D shows the fluorescent intensity against MSC adipoocyte culture for three donors.

FIG. 12 shows preferential adhesion of MSCs to superficial cartilage in culture medium compared to synovial fluid (n=6, *** indicates p=0.0007).

FIG. 13 shows the percentage of MSCs adhered to a fibrin clot over time relative to time=0 where cells are directly plated into a 24 well plate. Data is average plus range from at least four random fields.

FIG. 14 shows the number of MSC colonies formed following culture expansion over 14 days from brushing or mechanically agitating a small piece of human synovium with a variety of head designs with projecting elements of differing cross sectional diameters. The brush designs were cyto (cytology brush used as a control), C2 square shaped cross sectional area, C4 “V” shaped or chevron shaped cross sectional area, C6 a mix of C2 and C4, C7 diamond shaped in cross sectional area and C8 rectangular shaped in cross sectional area arranged in a broken chevron pattern so each neighbour is at right angles with respect to one another.

FIG. 15A shows the fold difference in DNA retained or released and FIG. 15B shows the percentage DNA released from a variety of head designs with projecting elements of differing cross sectional diameters (cyto, C2, C4, C6, C7 and C8).

FIG. 16 shows a comparative study of two head designs (C4 and C8) versus a cytology brush of DNA retained and released following brushing/mechanical agitation for three different synovial tissues.

FIG. 17A shows the total number of colonies obtained from cultures MSCs cells obtained from a synovial joint using a cytology brush from a washout, pre-brush and post-brush procedure during knee arthroscopy. FIG. 17B shows representative examples of cultured colonies for one patient from data presented in FIG. 17A

FIG. 18 illustrates one embodiment of the device of the present invention.

### DETAILED DESCRIPTION

Reference herein to an “abrasive surface” of the medical device of the present invention is synonymous with a “rough surface” and is intended to refer to a surface that has friction and includes a surface which has a plurality of abrasive projecting elements thereon in the form of, without limitation, a protuberance, fin, prong, hair, bristle, quill, fiber, rod or the like that, in use, is the surface of the medical device which contacts the synovial membrane tissue so as to activate or stimulate MSCs to release or to effect their migration to the site of injury and/or surgery.

Reference herein to a “non-abrasive surface” of the medical device of the present invention is intended to refer to a surface which lacks friction or has reduced friction or is substantially friction free and includes a surface which is not abrasive or rough, the surface ideally is smooth or flat and devoid of any projecting elements.

Reference herein to “improving surgical outcome” includes a rapid or improved recovery rate and/or an improvement in the recovery process as compared to a procedure carried out without the use of the medical device of the present invention. This may include improved healing time, a reduction in the likelihood of a repeat procedure, less reliance on physiotherapy and other forms of aftercare. By way of example, but not limitation, “improving surgical outcome” also includes improving the healing quality and/or reducing pain in the subject that has undergone the surgical procedure.

Reference herein to “synovial membrane” is synonymous with “synovium” or “stratum synoviale” and relates to the soft tissue found between the articular capsule (joint capsule) and the joint cavity of synovial joints, “adjacent tissue” includes the subsynovium and fatty or fibrofatty synovium of the fat pad closely juxtaposed to the synovium.

Reference herein to “released cells” includes the ability of cells to migrate or to be recruited to the site of stimulation or abrasion or injury of the synovial membrane. The device of the present invention releases MSCs from the synovial membrane or adjacent tissue giving them the opportunity to migrate and be recruited and adhere, stick or interact with the cartilage, ligament or meniscal tissue.

Reference here to “brushing” is intended to include any movement that mechanically agitates the surface of the synovial membrane without causing any significant damage to the membrane such as rupture or tears.

It is believed that synovial fluid MSCs (SF MSCs) are derived from both the synovial membrane and adjacent synovium and that the ready access of SF MSCs to cartilage and other joint tissues offers a novel strategy for joint repair. The intimate relationship between joint structures and synovium has led to the belief that the synovium may form a “tissue-specific niche” for MSCs. These synovium derived MSCs may already possess a strong bias towards intra-articular tissue repair, due to their shared environment and so many well be more able to respond most appropriately to tissue repair signals within the joint. It has been demonstrated that synovium-derived MSCs have superior chondrogenic capacity of MSCs from all mesenchymal tissues (Sakaguchi et al Arthritis and Rheumatism; 2005; (52(8):2521-9), cartilage engineered from synovium-derived MSCs have good mechanical properties (Ando et al Biomaterials; 2007; 28(36):5462-70) and have molecular profiles similar to those derived from SF (Sekiya et al J. Orthop. Res.; 2012; 30(6):943-949). Therefore, synovial manipulation offers a highly attractive strategy for joint repair. This is especially the case since the frequency of SF MSCs is low (Jones et al, Arthritis and Rheumatism, 2004; 50(3):817-27 and Jones et al Arthritis and Rheumatism 2008; 58(6): 1731-40).

It has been demonstrated in vitro that MSC liberation into joint fluid can be greatly increased by mechanical means and that fibrin clots entrapt such cells. This offers an ideal opportunity for translational cellular therapy in orthopaedic arthroscopy by bolstering endogenous MSCs in a cost effective way to repair joints by entrapment into the clot formed under microfracture.

The medical device of the present invention is intended to be used intra-operatively during arthroscopic procedures so as to stimulate the release of endogenous mesenchymal stem cells (MSCs) from synovial tissue, especially in the knee. The device is based around abrasive projecting elements design (but is not limited thereto), which will bolster numbers of MSCs present within the synovial fluid, increasing the endogenous reparative capacity of the synovial joint. It is envisaged that the device of the present invention can also be used alongside existing and future (such as the grafting of decellurlarised scaffolds) procedures for meniscal and ligament insufficiencies where MSC recruitment is needed. The device and methods of the present invention advantageously provide a cost effective and technically simple solution to allow the surgeon to increase MSCs numbers without the need to remove tissue for digestion, cell selection and culture expansion. The device represents a reliable and robust method for releasing and or delivering minimally manipulated autologous or allogeneic MSCs at an appropriate time and within the local environment where their regenerative capacity can be exploited.

The device of the present invention provides the first means to release or deliver intra-operative minimally manipulated autologous synovial MSCs without the need for expensive and time-consuming enzymatic release, cell separation or culture expansion. The device and procedure will enable the surgeon to reliably bolster stem cell numbers within the joint allowing direct access of these MSCs to injured joint structures. The device is designed to integrate into existing arthroscopic procedures, ensuring ease of use with minimal increase in operative times.

The device of the present invention comprises a head region with a plurality of projecting abrasive, flexible elements specifically designed and constructed for use during arthroscopic procedures. In one embodiment, is intended that the surgeon performs a routine arthroscopic investigation to repair cartilage, ligament or meniscus with a final short procedure (typically less than 5 minutes) using the device to brush the synovium. This brushing action releases cells and small fragments of tissue into the knee or whatever other synovial joint is being operated on, where MSCs within the release component will have direct access to the damaged joint structures. At the end of the procedure the device is removed and the knee or other joint is closed, entrapping the MSCs within the joint.

The device of the present invention bolsters the number of MSCs within synovial fluid by gentle manipulation of the joints synovial lining using the device of the present invention. During arthroscopic surgery one of the first procedures is to irrigate the knee with saline which causes a loss of any synovial fluid MSCs already present. Using an incision (with or without a cannula), the surgeon can introduce the device of the present inventioninto the joint cavity (without irrigation). Under arthroscopic guidance the surgeon can then agitate the synovium (either manually or by an applied mechanical vibrational force) thus dislodging and releasing MSCs into the joint.

Part of the inventive nature of the device of the present invention resides in the discovery that the cross-sectional shape of projecting elements makes a substantial and material difference to the ability to release/stimulate MSCs to a surgical site during a procedure and therefor obviates the need for extra corporeal enrichment. The present invention has demonstrated in particular that non-circular cross sectional projecting elements have a greater ability to achieve this potential.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

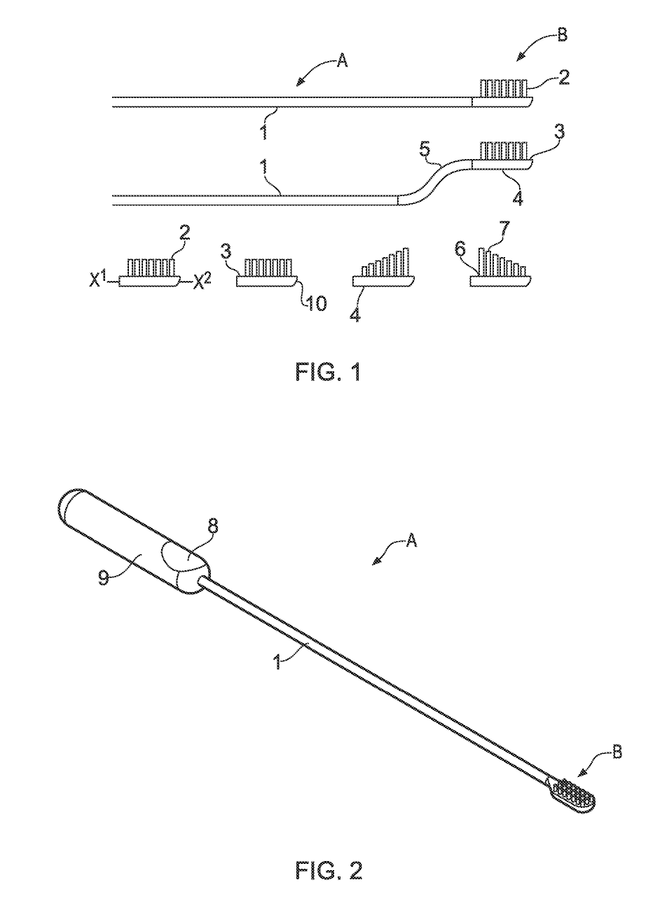

The reader's attention is directed to all papers and documents which are filed concurrently with or previous to this specification in connection with this application and which are open to public inspection with this specification, and the contents of all such papers and documents are incorporated herein by reference. FIG. 1 shows the medical device of the present invention (A) comprising a head region (B) with a rounded or profiled distal end (10). The head region has an abrasive surface (3) and an opposing non-abrasive or smooth surface (4). The neck portion (B) is attached to a stem portion (1) and maybe angled at a neck portion with respect to the stem region (5). Associated with the abrasive surface (3) are a plurality of projecting elements (2), these projecting elements maybe associated with the abrasive surface by either being formed integrally therewith, moulded thereto or attached or adhered thereto. The connection to the abrasive surface is by their base (6). The projecting elements may be rigid or flexible or a mixture of both along their length and may move about their base (6). The length of the projecting elements from tip (7) to base (6) may be either uniform from length (X⁻ to X²). In some embodiments the projecting elements (2) along length (X¹ to X²) are tapered so that they may be either longer at either the distal or proximal end, reducing in length to the proximal or distal end respectively or they may be shorter at both distal and proximal ends tapering to a peak at a point between length (X¹ to X²). FIG. 2 shows one embodiment of the medical device (A) of the present invention, including the head portion (B), guide portion (1) with a notch (8) in the handle portion (9). The notch (8) allows the surgeon to ascertain where the head region is in relation to the synovial tissue in the patient when inserted into a patient and acts as an orientation guide when the device is in use.

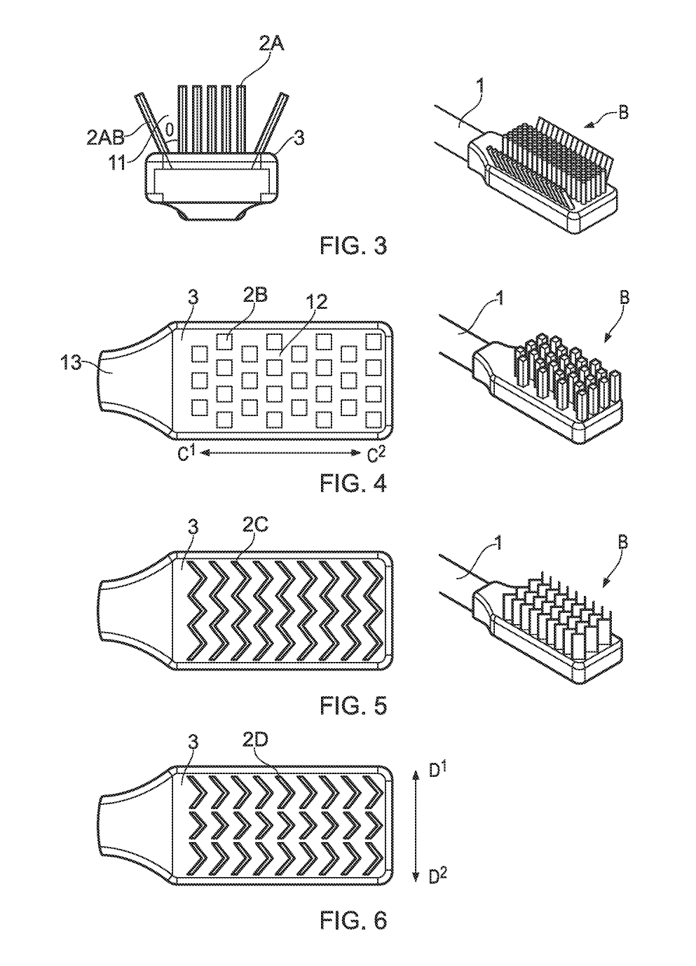

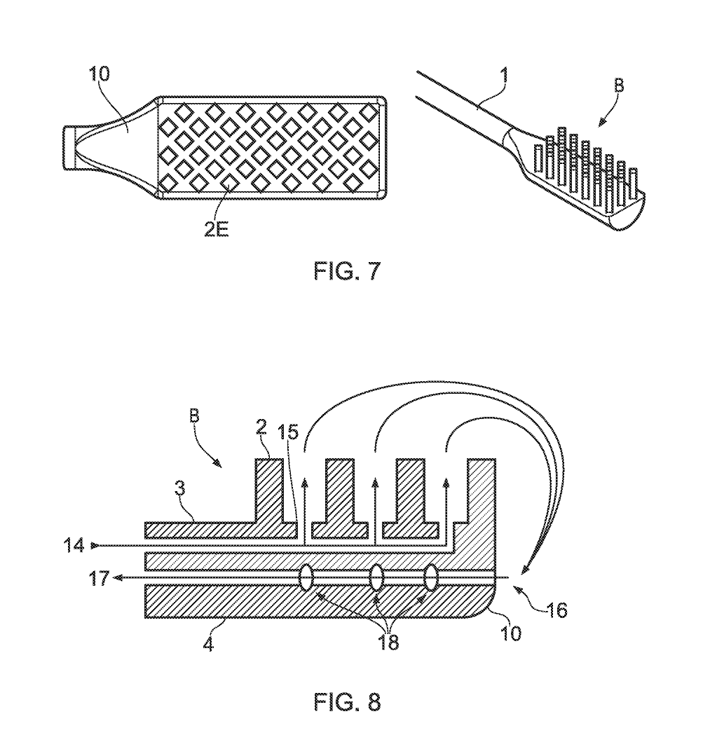

With reference to FIGS. 3 to 7 there are shown alternative embodiments of different cross-sectional shapes of the projecting elements. FIG. 3 shows projecting elements (2A) of head portion (B) attached to the guide portion (1) by an attachment (13), the projecting elements are in the form of perpendicular fibre bundles with peripheral bundles (2AB) angled outward to form gaps (11). FIG. 4 shows projecting elements (2B) which are square in cross-section with gaps (12) formed between columns (C¹ to C²) and rows (D¹ to D²). The numbers of columns and rows is variable and dependent on what type of synovial joint is being considered, the numbers are not intended to limit the scope of the invention. FIG. 5 shows a number of projecting elements (2C) in the form of a contiguous wave, whereas FIG. 6 shows projecting elements (2D) in the form of discrete separated “V” shapes or chevrons. FIG. 7 shows the projecting elements (2E) of cross-sectional shape in the form of diamonds or rhomboids. It will be appreciated that the head portion or region may comprise a number of projecting elements of a single particular design or cross-sectional shape arranged in different surface patterns on the abrasive surface, they may also be of a mixed cross-sectional shape and preferably at least a proportion are in the form of diamonds or rhomboids as depicted in FIG. 7 or rectangles (C8).

FIG. 8 shows an alternative of the head region (B) of the medical device of the present invention in which the head region is provided with an irrigation channel (14) and an aspiration channel (17) positioned between the abrasive surface (3) and non-abrasive surface (4). The irrigation channel is provided with a number of irrigation ports (15) positioned between projecting elements (2) so that pressurised fluid can be passed between the projecting elements into the synovial cavity. This provides the added benefit of removing any potentially trapped cells. The aspiration channel (17) is under suction pressure and collects fluid from the surrounding area through an aspiration port (16) and any other aspiration ports (18) positioned along device head. Thus the device can irrigate and aspirate simultaneously, so that cellular material expelled from the device head can be removed from the joint via an incorporated aspiration port and channel. In another embodiment of the invention the irrigation channel maybe provided where the aspiration channel is shown in FIG. 8 so that aspiration occurs between the projecting elements and the irrigation is via the side and end aspiration ports of FIG. 8. Alternatively aspiration could be via an additional instrument so that the head region (B) comprises only an irrigation channel and associated ports. In another embodiment the irrigation could be by a separate additional instrument so that the head region (B) comprises only an aspiration channel and associated ports. Cellular material can then be captured, concentrated, filtered or selected (for inclusion or exclusion of specific cell types) and reintroduced via injection or loading onto scaffold (in appropriate scaffold for cartilage, meniscus, ligament).

### EXAMPLE 1

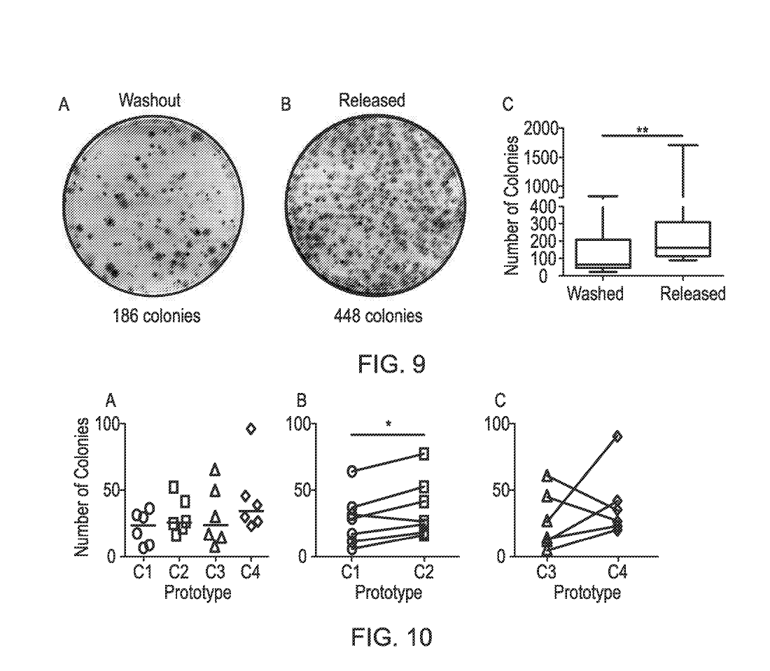

Synovial compartments of cadaveric knees were washed out using 50 mL phosphate buffered saline (PBS) to determine number of MSCs within the synovial fluid prior to release from synovium. After this a further 50 mL PBS was injected into the knee cavity. Through a small incision a brush with a number of projecting elements attached to a motorised drill for increased abrasion was inserted and used to agitate the superficial synovium. The 50 ml of PBS containing released cells were aspirated from the joint and cells within this and the initial washout were grown for 14 days to produce colonies (the so called colony forming unit-fibroblastic, CFU-F assay, FIG. 9). Results show that FIG. 9A had substantially less colonies (186) as compared to FIG. 9B which had 448 colonies from the stimulated synovial tissue and released MSCs. This was a significant difference (p=0.002) and showed that contacting the synovium with an abrasive surface and agitation the abrasive surface of the device against the synovium causes the release of MSCs.

### EXAMPLE 2

Synovial membrane (synovium) was taken from patients undergoing total knee replacement surgery. After washing of the membrane to remove loosely bound cells, the synovium was held in place using forceps. The devices (C1 with circular cross-sectional projecting elements; C2 with square cross-sectional projecting elements; C3 with continuous wave projections and C4 with “V” shaped or chevron cross-sectional projecting elements) were applied to the surface of the synovium using downward pressure and vertical stokes to detach superficial cells (including MSCs). The synovium was then washed to remove any remaining detached cells. These cells were then grown for 14 days in a CFU-F assay. The colonies were fixed in formalin, stained and counted (FIG. 10A). The comparative results (FIG. 10B device C1 versus C2 and FIG. 10C devices C3 and C4) showed that in order of performance, based on the number of colonies, that of the projecting elements the circular cross-sectional projecting elements of the four designs tested was the least effective but nonetheless all devices released MSCs.

### EXAMPLE 3

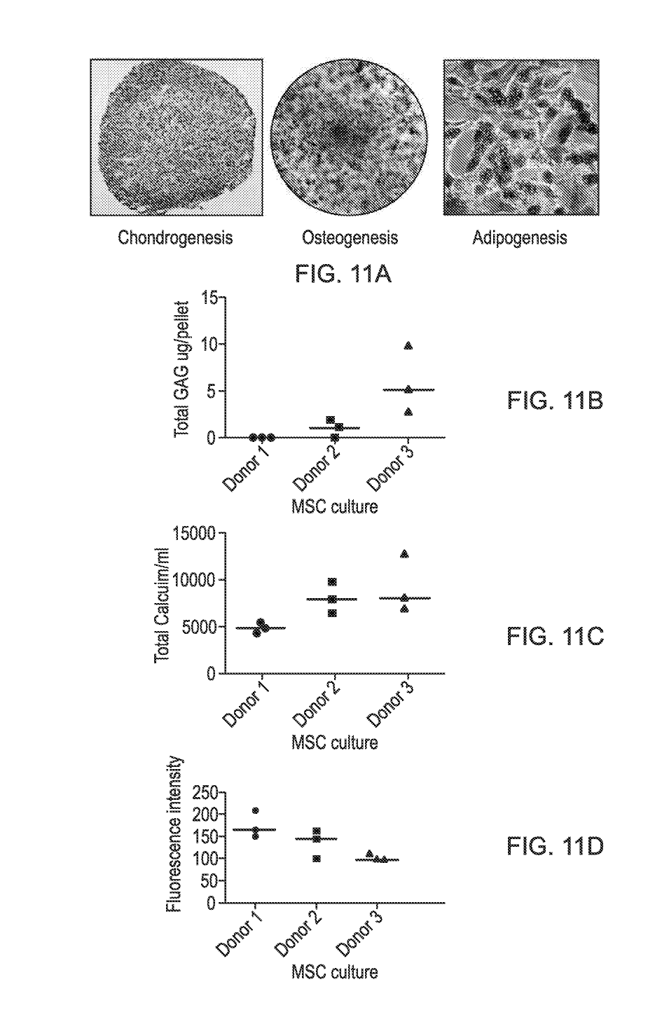

MSCs are thought to be key cellular mediators for the repair bone and cartilage by their ability to differentiate into mesenchymal tissue such as bone, cartilage and fat. The ability of the device of the present invention to release MSCs from the synovium is shown in FIG. 9 by the formation of colonies in the CFU-F assay. To further demonstrate these cells are MSCs and to show their ability to form tissue of mesenchymal origin, tri-lineage differentiation was performed on culture expanded cells released from human synovium by the intended device. FIG. 11A shows the ability of the released cells to form cartilage (chondrogenesis), bone, (osteogenesis) and fat (adipogenesis), further confirming the cells as MSCs.

The present invention comprises an orthopaedic device capable of detaching and releasing superficial cellular material from the synovium membrane of articulating joints. This cellular material contains viable MSCs as characterised by colony forming ability (FIG. 9) and differentiation capacity into tissues of mesenchymal lineage (cartilage, bone and fat, FIG. 11A-D). Several different embodiments show improved release of cells, in particular the embodiment depicted in FIGS. 6 shows improvement over that of circular or stellate cross-sectional projecting elements. In a group comparison the “V” shaped cross sectional projecting elements perform well however when comparing matched samples to a cytology brush the C8 design (rectangular cross sectional area where neighbours are at right angles with respect to one another) this appears to be the most effective design. This data together with the tri-lineage differentiation data as shown in FIGS. 11B-D demonstrates an improved release of cellular material compared to cellular material entrapped/retained within the head portion.

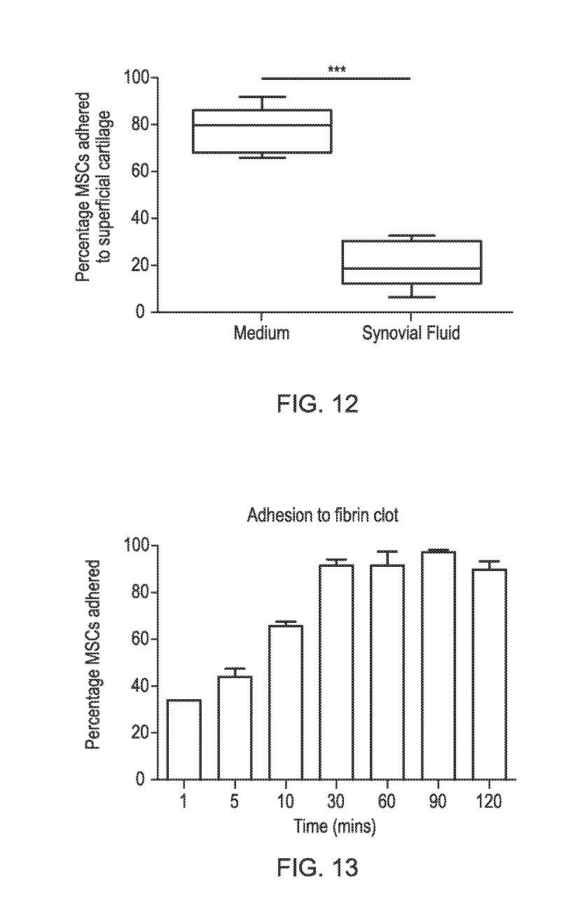

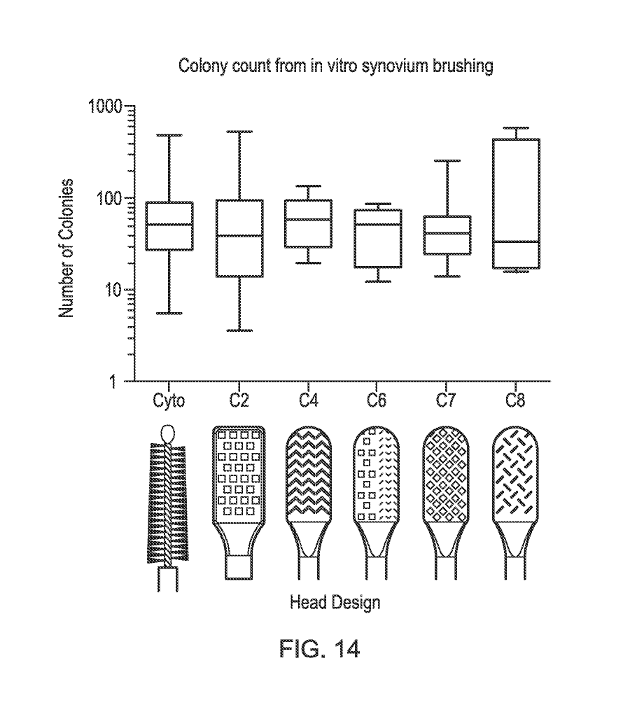

The release of cellular material using the device of the present invention is particularly an advantage during arthroscopic procedures. Under this procedure and using the methods of the present invention, synovial fluid contained within the joint and any resident MSCs are lost due to irrigation of the joint. This loss of synovial fluid and the replacement of the cells within the joint by the present invention is further supported by data showing MSCs are inhibited from adhering to cartilage due to properties of the synovial fluid (FIG. 12). The data from FIG. 12 represents a scenario analogous to that in arthroscopy whereby the synovial fluid is removed together with any resident MSCs by joint irrigation. The methods of the present invention will therefore replace and bolster these lost cells in a synovial fluid free environment where they can more readily adhere to superficial cartilage.

### EXAMPLE 4

It is anticipated in one instance that this device could be used in conjunction with a surgical procedure known as microfracture, whereby the subchondral bone is breeched in order to induce bleeding and clot formation. This procedure is used to treat cartilage defects and it is thought MSCs from bone marrow are entrapped within the clots, which subsequently forms repair tissue. The device described will reliably increase the number of MSCs present within the joint, where these MSC will migrate and integrate into the clot formed under microfracture. To determine if MSC released into the synovial fluid could participate in repair, their ability to adhere to clots was examined (FIG. 13). Clots were formed using bovine fibrinogen (so called fibrin glue).

One way in which this might be accelerated/facilitated is if the site of injury contains a blood clot (or marrow clot in the case of subcondral bone drilling and microfracture) or fibrin glue. The initial stage of this integration involves MSC adhesion to the clot surface.

We have tested MSC adhesion to a fibrin clot and found MSCs rapidly adhered to the fibrin surface, within 30 mins. FIG. 13 shows that maximum adhesion was achieved in as little as 30 minutes

MSCs were allowed to interact with a fibrin clot for up to 2 hours. Fibrin clots were formed in the lid portion of a 1.5 mL microfuge tube before the addition of a suspension of 10,000 MSCs. The tubes inverted so that the cells came to rest on the clot surface under gravity. At 30 minute intervals the microfuge tubes were again inverted and the cell suspension added to the well of a 24 well plate to adhere overnight before being fixed and stained with methylene blue. The number of cells that adhered to the well (and hence the number of cells that remained in suspension and did not adhere to the clot) were counted in four random fields from each time point. Within 30 minutes, 90% of all MSCs had adhered to the clot surface. This was maintained up to 2 hours (FIG. 13). It has also been demonstrated that released synovial MSCs migrated better into platelet poor plasma clots than into platelet rich plasma clots (data not shown).

In conclusion it has been shown that synovial MSCs can be released by mechanical means and were capable of migrating into the clots in vitro, which was dependent on clot composition. The study findings indicate that novel cartilage regenerative strategies using MSCs can be employed within orthopaedics. This work represents a bench to operating theatre strategy, to augment joint repair in a one stage cost effective procedure, based on knowledge of in vivo joint MSCs

### EXAMPLE 5

Results from a compartaive study of a cytology brush versus head designs C2, C4, C6, C7 and C8 tested using synovium from total knee replacements are shown in FIG. 145 Data shows the number of MSC colonies formed following culture expansion over 14 days after brushing a small piece of human synovium. Head design C8 was found to be the most effective, design C8 comprises a number of rectangular projections off set at rightangles with respect to their neighbours in a broken chevron pattern

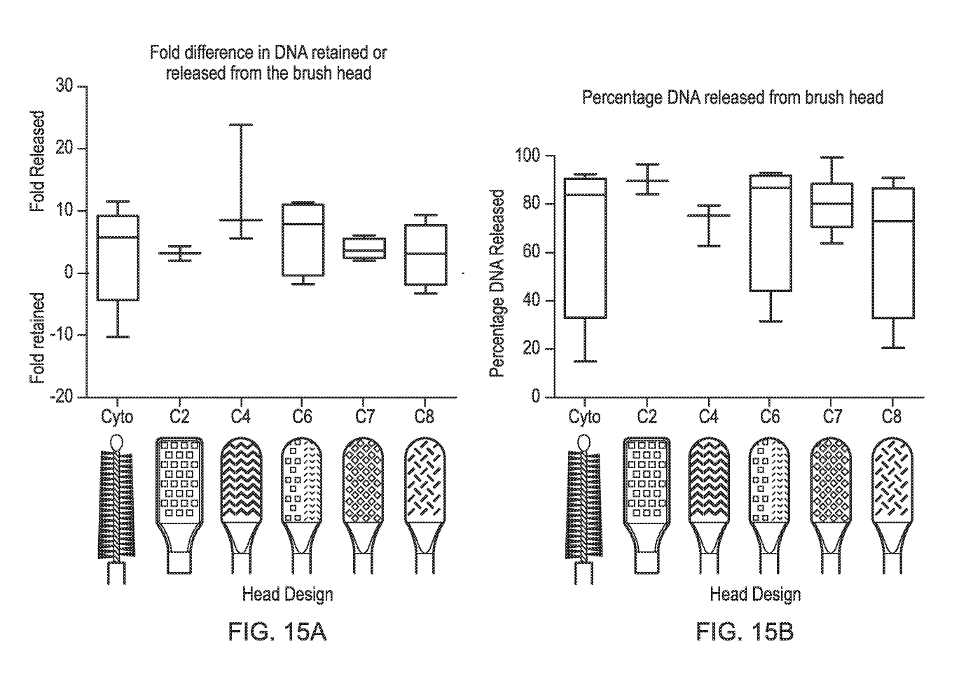

DNA content of the cellular material released from the synovium by the various head designs were measured and compared to the amount of DNA retain on each head design. This data indicates how much cellular material is trapped on the head and how much cellular material is released. This is shown in FIG. 15A as the difference in DNA released from the brush or DNA retained on the brush as a fold change (positive numbers means more DNA was released than retained, negative means more was retained than released). Alternatively, this can be view as the percentage of DNA released from the brush head (DNA released from the brush as a percentage of the total, retained and released), FIG. 15B. As is apparent from the Figures the cytology brush retains more DNA than the head designs of the present invention which is to be expected since the purpose of a cytology brush is to trap cells rather than with the present invention released them from synovial fluid. Table 1 below shows the values as means±S.E.M in brackets.

TABLE 1

| | Cross- Sectional Shape | | | | | |
|---|---|---|---|---|---|---|
| | | | Mixed Square-V | | | |
| | Square | V shaped | shaped | Diamond | Rectangle | Cytology |
| | (C2) | (C4) | (C6) | (C7) | (C8) | (stellate) |
| Fold Release | 2.85 (0.63) | 12.20 (5.6) | 5.93 (3.05) | 2.8 (2.3) | 2.65 (2.54) | 2.77 (2.7) |

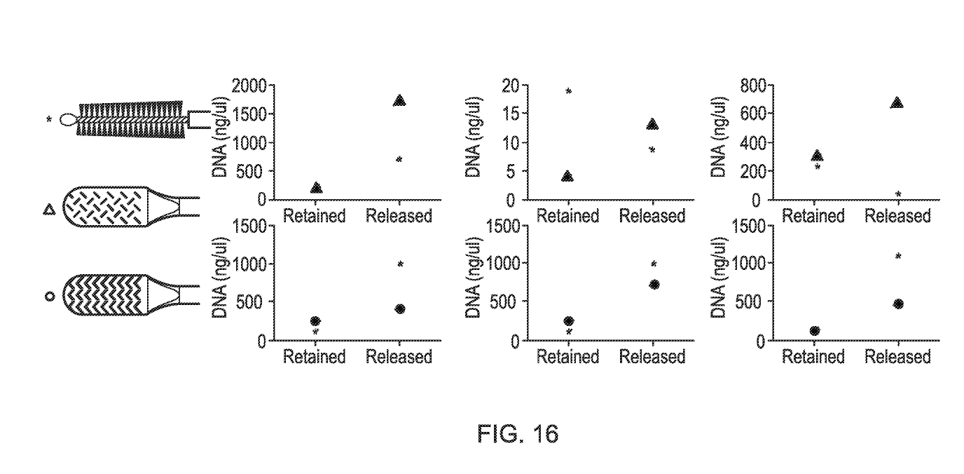

A comparison of the DNA retained/released for C8 with the matching patients' synovium also used with the cytology brush and C4 design (FIG. 16) shows that C8 appears to be out performing the cytology brush in terms of DNA released from the brush head (in three out of four experiments). On the other hand, direct comparison of matching samples for cytology and C4 shows that the cytology brush is better. This data also highlights the patient variability which may account for the wide spread in the above data.

### EXAMPLE 6

The durability of the various head designs were tested by simulating its use in a porcine knee model. Three examples of three different designs were used alongside three cytology brushes to continuously brush the porcine synovium for five minutes. Five minutes exceeds the amount of time the brush would be used in situ in humans. Results (data not shown) indicate, with the head designs of the present invention, no damage was seen after their use. For the cytology brush however, one of the three used was difficult to introduce into the knee and bent up on insertion and one was bent up on removal from the knee. These observations are consistent with the surgeon's experience with the cytology brush during arthroscopy.

### EXAMPLE 7

Clinicians' reports following the use of a cytology brush during arthroscopy expressed concern with the flexibility of the cytology brush, noting that on several occasions the wire portion of the brush which holds the bristles has bent upon insertion into the knee (. This is a particular problem in patients with more subcutaneous fat, as there is more potential for the brush head to get “snagged” in tissue before entering the knee. In many cases the cytology brush has to be introduced through a cannula often using forceps. Clinicians were concerned that the brush head could break off if it bents too much. In addition to this flexibility increasing the chance of brush failure, the flexibility also reduces the amount of pressure that can be applied to the synovium. This results in the surgeon having less “feel” and confidence in the positioning and motion of the brush head during use, making it difficult for them to assess the amount of force they are applying to the synovium. The cytology brush is also too short in length to adequately brush the majority of the synovium. Access is limited to a small area of either the medial or lateral gutter. Due to the concerns with the flexibility of the cytology brush clinicians did not want to risk introducing this brush a second time to the remaining gutter which would increase stem cell yield.

In addition , it has been noticed that in many cases after using the cytology brush on the small area of synovium that is accessible, that the bristles become clogged/matted with cellular material. It is likely that once the bristles are clogged/matted its effectiveness is reduced. This feature is in-keeping with the intended use of this device, i.e. to capture cells and remove them from the body.

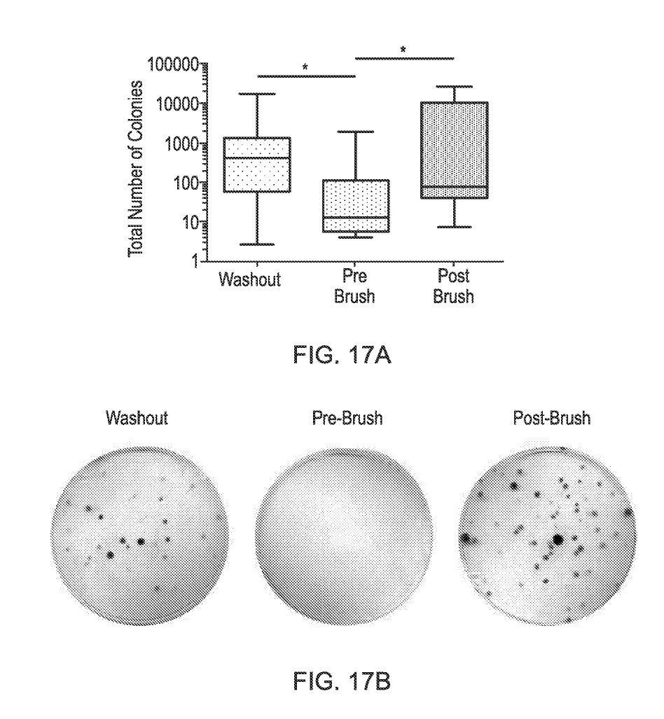

FIG. 17A shows the total number of MSC colonies following the use of the cytology brush during arthroscopy (n=9, with an example from one donor shown in FIG. 17B). This data highlights how the arthroscopy procedure results in the loss of the naturally occurring stem cells in the joint (represented by the colonies in the ‘Washout’ column). The ‘Pre-Brush’ column represents the number of MSCs that would be present at the end of the arthroscopy without the use of the brush. The ‘Post-Brush” column shows that these can be replaced by synovial brushing. This number could be greatly improved using a purpose build device of the present invention that can reach more of the synovium surface.

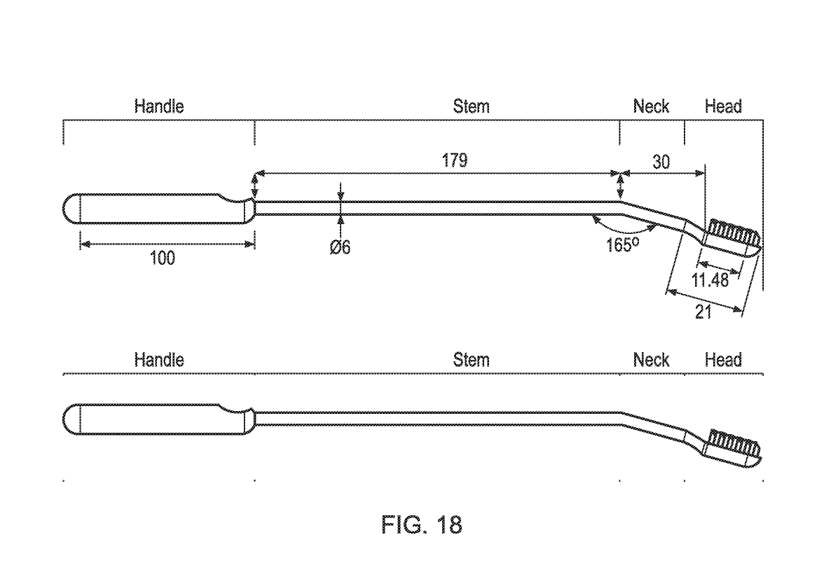

Following the clinicians' experience, the device shown in FIG. 18 is an ideal embodiment of the present invention which will allow sufficient length of the device to increase the range and easy of synovium access, particularly in the supra-patellar pouch which has a large area of synovium in the knee. Would meet all flexibility issues and provides a head design with maximum capacity to stimulate MSC cell release.

1. An orthopaedic medical device comprising a head portion, a neck portion, a stem portion and a handle portion, the head portion having a first surface that is abrasive and provided with at least one abrasive, flexible projecting element that is non-circular in cross section and an opposing second surface, the second surface being non-abrasive or smooth.

2. A device according to claim 1 comprising a plurality of projecting elements each element having a tip and a base.

3. A device according to claim 1, wherein the projecting elements are either formed integrally at their bases with the abrasive surface of the head portion or moulded thereto or attached thereto at their bases.

4. A device according to claim 1, wherein the projecting elements are flexible or rigid or comprise both a flexible part and a rigid part.

5. A device according to claim 1, wherein the projecting elements are moveable about their base.

6. A device according to claim 1, wherein the projecting elements are in the form of a bristle, fin, prong, hair, spike, quill, fiber or rod.

7. A device according to claim 1, wherein the projecting element tip is flat, tapered, conical or profiled.

8. A device according to claim 1, wherein the projecting elements are perpendicular with respect to the abrasive surface of the head portion.

9. A device according to claim 1, wherein a proportion of projecting elements are perpendicular with respect to the abrasive surface of the head portion and a proportion of projecting elements are angled with respect to the abrasive surface between 35° to less than 90° with respect to the abrasive surface of the head portion.

10. A device according to claim 1, wherein the projecting elements are arranged in columns and rows on the abrasive surface.

11. A device according to claim 10 wherein the number of columns is between 2 to 20.

12. A device according to claim 10 wherein the number of rows is between 2 to 10.

13. A device according to claim 1, wherein the projecting elements in cross section are shaped as squares, rectangles, diamonds, circles, triangles, stellate (star-shaped), rhombic, oval, chevrons, “V” shaped, “W” shaped, pentagons, hexagons or other multiple sides shapes.

14. A device according to claim 1, wherein the abrasive surface comprises projecting elements that are all of the same cross-sectional shape or a variety of two or more mixed cross-sectional shapes.

15. A device according to claim 1, wherein at least a proportion of the projecting elements are rectangular, diamond or rhombic shaped in cross section.

16. A device according to claim 1, wherein the projecting elements are:
(i) all of equal length;
(ii) of a greater length at a proximal end of the abrasive surface graduating to a shorter length at a distal end of the abrasive surface;
(iii) of a greater length at a distal end of the abrasive surface graduating to a shorter length at a proximal end of the abrasive surface;
(iv) of greater length at both a distal and proximal end graduating to a shorter length at a position therebetween; or (v) of shorter greater length at both a distal and proximal end graduating to a peak of greater length at a position therebetween.

17. A device according to claim 1, wherein the density of the projecting elements is in the range from about 1 to 1000 projecting elements/cm².

18. A device according to claim 1, wherein the total distance of the underside or base of the non-abrasive side of the head portion to the tip or apex of the projecting element is between 3.00 to 20.00 mm, and more preferably is between 6.00 to 12.00 mm and more preferably still is between 7.00 to 10.00 mm or any tenth or one hundredth integer of 0.00 to 0.99 and therebetween.

19. A device according to claim 1, wherein the length of the projecting element from base to tip is in the range from about 1.00 mm to about 5.00 mm or any tenth or one hundredth integer of 0.00 to 0.99 and therebetween.

20. A device according to claim 1, wherein the head portion, neck portion, stem portion and handle portion are constructed of the same or of different materials.

21. A device according to claim 1, wherein the projecting elements are constructed of any one or more of the following materials selected from the group comprising acetal, Teflon (polytetrafluoroethylene), polyester, nylon, polyethylene, polyurethane, polypropylene, polycarbonate, polyether ether ketone (PEEK) or any combination thereof.

22. A device according to claim 1, wherein the length of the head portion is between 5 to 25 mm in length.

23. A device according to claim 1, wherein the head portion, neck portion, stem portion are formed integrally or wherein the each portion is detachable and/or disposable.

24. A device according to claim 1, wherein the distal tip of the head portion is curved.

25. A device according to claim 1, wherein the stem portion is between 100 to 500 mm in length.

26. A device according to claim 1, wherein the neck portion is angled with respect to the stem portion.

27. A device according to claim 26 wherein the neck portion is angled with respect to the horizontal axis of the stem portion by between 5 to 30°.

28. A device according to claim 1, wherein the head portion of the medical device of the present invention is provided with one or more additional channels for the delivery of a fluid to a surgical site and/or suction of fluid therefrom.

29. A device according to claim 28 wherein at least one of the additional channels is pressurized so as to deliver fluid for irrigation and at least one other channel is under vacuum for aspirating liquid and cellular debris away from the site.

30. A device according to claim 29 wherein the irrigation channel and/or aspiration channel is provided with a number of ports adjacent the base of the projecting elements.

31. A device according to claim 1, wherein the stem portion includes a marker region to assist in orientation of the medical device in situ and/or to ascertain when the head portion is in position at the appropriate surgical site.

32. A device according to claim 1, including an external power source that provides vibrational or oscillatory movement to the head region.

33. Use of the device according to claim 1, in an arthroscopic procedure.

34. An arthroscope or other synovial joint seeping device including the device according to claim 1.

35. A method of stimulating release of mesenchymal stem cells (MSCs) cells and/or encouraging migration of mesenchymal stem cells (MSCs) and/or recruiting mesenchymal stem cells (MSCs) in situ to a surgical site or site of injury during an operative procedure on a synovial joint, the method comprising contacting synovial membrane tissue with a device having at least one abrasive surface.

36. The method of claim 35, wherein the device is an orthopaedic medical device comprising a head portion, a neck portion, a stem portion and a handle portion, the head portion having a first surface that is abrasive and provided with at least one abrasive, flexible projecting element that is non-circular in cross section and an opposing second surface, the second surface being non-abrasive or smooth.

37. The method of claim 35, wherein the synovial joint is selected from the group comprising gliding joints, hinge joints, pivot joints, condyloid joints, ball and socket joints or compound joints.

38. The method according to claim 35, wherein the synovial joint is a compound joint and optionally is a knee joint.

39. The method according to claim 35, wherein the operative procedure is selected from the group comprising arthroscopy, meniscectomy, chondroplasty, cruciate ligament repair, knee replacement, mosaicaplasty meniscus repair.

40. The method according to claim 35, wherein the operative procedure is knee arthroscopy and is conducted for the purposes selected from the group comprising:
(i) to remove or repair torn meniscal cartilage which cushions the space between the bones in the knee;
(ii) to reconstruct a torn anterior cruciate (ACL) or posterior cruciate ligament (PCL);
(iii) to trim torn pieces of articular cartilage;
(iv) to remove loose fragments of bone or cartilage;
(v) to remove inflamed synovial tissue;
(vi) to repair misalignment of the patella;
(vii) to aid repair of osteochondral defects including osteochondritis dessicans;
(viii) to treat arthritis in younger patients;
(ix) for diagnostic or investigational purposes;
(x) mosiacaplasty grafting by transferring one or more cylindral osteochondral autografts from a low weight-bearing area of the knee towards the defective site; or
(xi) to collect allogeneic MSCs from a donor for subsequent implantation into a recipient.

41. A method of increasing a cell population of mesenchymal stem cells (MSCs) in situ at a surgical site or injury site during an operative procedure on a synovial joint, the method comprising contacting synovial membrane tissue with a device having at least one abrasive surface.

42. The method of claim 41, wherein the device is an orthopaedic medical device comprising a head portion, a neck portion, a stem portion and a handle portion, the head portion having a first surface that is abrasive and provided with at least one abrasive, flexible projecting element that is non-circular in cross section and an opposing second surface, the second surface being non-abrasive or smooth.

43. A method of delivering intra-operative minimally manipulated autologous mesenchymal stem cells (MSCs) to a surgical site or site of injury during an operative procedure on a synovial joint, the method comprising contacting synovial membrane tissue with a device having at least one abrasive surface.

44. The method of claim 43, wherein the device is an orthopaedic medical device comprising a head portion, a neck portion, a stem portion and a handle portion, the head portion having a first surface that is abrasive and provided with at least one abrasive, flexible projecting element that is non-circular in cross section and an opposing second surface, the second surface being non-abrasive or smooth.

45. A method of improving surgical outcome of a synovial joint surgical procedure, the method comprising contacting synovial membrane tissue with a device having at least one abrasive surface during the surgical procedure.

46. The method of claim 45, wherein the device is an orthopaedic medical device comprising a head portion, a neck portion, a stem portion and a handle portion, the head portion having a first surface that is abrasive and provided with at least one abrasive, flexible projecting element that is non-circular in cross section and an opposing second surface, the second surface being non-abrasive or smooth.

47. A method of delivering intra-operative minimally manipulated allogeneic mesenchymal stem cells (MSCs), collected from a donor, to a surgical site during an operative procedure on a synovial joint, the method comprising contacting synovial membrane tissue of an allogeneic donor with a device having at least one abrasive surface and collecting said cells.

48. The method of claim 47, wherein the device is an orthopaedic medical device comprising a head portion, a neck portion, a stem portion and a handle portion, the head portion having a first surface that is abrasive and provided with at least one abrasive, flexible projecting element that is non-circular in cross section and an opposing second surface, the second surface being non-abrasive or smooth.

49. A method of delivering intra-operatively a concentrate of minimally manipulated autologous mesenchymal stem cells (MSCs) collected from a patient to a surgical site during an operative procedure on a synovial joint, the method comprising contacting synovial membrane tissue with a device having at least one abrasive surface and collecting said cells, preparing a concentrate of said cells and re-introducing said cell concentrate to the surgical site.

50. A method of delivering intra-operatively a concentrate of minimally manipulated allogeneic mesenchymal stem cells (MSCs) collected from a donor, to a surgical site during an operative procedure on a synovial joint, the method comprising contacting synovial membrane tissue of the donor with a device having at least one abrasive surface and collecting said cells, preparing a concentrate of said cells and introducing said cell concentrate to the surgical site of a recipient.

51. The methods of claim 35, wherein the device is an orthopaedic medical device comprising a head portion, a neck portion, a stem portion and a handle portion, the head portion having a first surface that is abrasive and provided with at least one abrasive, flexible projecting element that is non-circular in cross section and an opposing second surface, the second surface being non-abrasive or smooth.

52. The method according to claim 35, further including the step of collecting released MSCs and forming a concentrate therefrom and re-introducing said concentrate into a patient during the operative procedure.

53. The method of claim 35, including the step of microfracture to induce a clot.

54. A method of augmenting a microfracture procedure comprising delivering intraoperatively a concentrate of minimally manipulated autologous mesenchymal stem cells (MSCs) collected from a patient to a clot formed by the microfracture during an operative procedure on a synovial joint, the method comprising contacting synovial membrane tissue with a device having at least one abrasive surface and collecting said cells, preparing a concentrate of said cells and re-introducing said cell concentrate to the microfracture clot site.

55. A method of augmenting a microfracture procedure by increasing a cell population of mesenchymal stem cells (MSCs) in situ at a microfracture site, the method comprising contacting synovial membrane tissue with a device having at least one abrasive surface.

56. Use of allogeneic MSCs in recellularising a native or synthetic scaffold for subsequent implantation into a recipient.

57. Use according to claim 55 wherein the scaffold is an implantable device for use inside, adjacent or external to a synovial joint or for ligament and meniscal repair.
